(19) United States
(12) Patent Application Publication
(10) Pub. No.: US 2005/0176144 A1
(43) Pub. Date: Aug. 11, 2005

(54) Culture media compositions free of fetal bovine serum

(76) Inventor: Jose A. O'Daly, Florham Park, NJ (US)

Correspondence Address: BOND, SCHOENECK & KING, PLLC, ONE LINCOLN CENTER, SYRACUSE, NY, 13202-1355, US

(21) Appl. No.: 10/773,578

(22) Filed: Feb. 6, 2004

(51) Int. Cl. C12N 5/02; C12N 1/20

(52) U.S. Cl. 435/404; 435/252.1; 435/383

(57) ABSTRACT

A cell culture growth media free of Fetal Bovine Serum for use with parasitic organisms. The media includes calcium chloride, sodium bicarbonate, potassium chloride, sodium chloride, monosodium phosphate, glucose, hepes, ferric nitrate, magnesium sulfate, tricine, d-ribose, 2-deoxy ribose, adenosine-5-triphosphate (ATP), 2-deoxyadenylic acid (d-AMP), 5′-thymidylic acid (TMP), 2′-deoxyicitidine-5 monophosphate (d, 2′-deoxyuridine-5-monophosphate (d, 2′-deoxyguanilic Acid (d-GMP), aspartic acid, glutamic acid, 1-alanine, arginine, carnosine, cysteine, cystine, glutamine, glycine, histidine, iso-leucine, leucine, lysine, methionine, ornitine, phenylalanine, proline, serine, threonine, tryptophan, tyrosine, valine, ascorbic acid, biotine (H), carnitine, cholecalciferol, choline chloride, cyanocobalamine (B₁₂), ergocalciferol, folic acid, myo-inositol, menadione, nicotinamide, PABA, panthotenato, pyridoxal, pyridoxamine, pyridoxine, retinol (A), riboflavine (B₂), Thiamine (B₁), 6,8 Thiotic acid, alfa-tocoferol, 3-phytylmenadione (K₁), tetrahydrofolic acid, hemin from procine, and nanopure water.

### FIELD OF THE INVENTION

The present invention relates generally to a growth medium for biologics. More specifically, the invention relates to a growth medium that is free of fetal bovine serum and is used for growth of parasitic organisms.

### BACKGROUND

Scientists and researchers have used various types of media to support cultivation of different types of cell lines. The composition of the cell growth media is important because the composition effects cell survival and cellular response to many influences. Sera derived from bovine sources have been used as a nutrient and as a hormonal source for cell culture media. Fetal Bovine Serum (FBS) may vary in composition, hormone content, and contaminants. Recent cases of bovine spongiform encephalopathy, or mad cow disease, have increased the need to develop alternatives to the use of FBS in many cell growth media.

There have been alternatives proposed to the use of FBS in some types of growth media. However, serum-free media are highly specific to a particular cell type or contain components extracted from serum. One such example is found in U.S. Pat. No. 6,617,161 to Luyten et. al which describes a serum-free cell growth medium that is useful in the cultivation of cartilaginous phenotypes in chondrocytes.

In many instances, scientists require a growth medium to support growth in various and specific types of biologics. One area of particular interest is in the growth of parasitic organisms, for example, the cultivation in the amastigote stage of Leishmania genus. This is of particular importance in the production of compositions for the treatment of psoriasis, leishmaniasis, and possibly other immunologic disorders. Methods of producing immunotherapeutic agents from the amastigote stage of Leishmania genus are described in U.S. Pat. No. 6,673,351 to O'Daly. There is a particular need for a FBS-free growth medium for use with parasitic organisms.

### SUMMARY OF THE INVENTION

The present invention provides a FBS-free growth medium for use with parasitic organisms. The medium includes calcium chloride, sodium bicarbonate, potassium chloride, sodium chloride, monosodium phosphate, glucose, hepes, ferric nitrate, magnesium sulfate, tricine, d-ribose, 2-deoxy ribose, adenosine-5-triphosphate (ATP), 2-deoxyadenylic acid (d-AMP), 5′-thymidylic acid (TMP), 2′-deoxyicitidine-5 monophosphate (d, 2′-deoxyuridine-5-monophosphate (d, 2′-deoxyguanilic Acid (d-GMP), aspartic acid, glutamic acid, 1-alanine, arginine, carnosine, cysteine, cystine, glutamine, glycine, histidine, iso-leucine, leucine, lysine, methionine, ornitine, phenylalanine, proline, serine, threonine, tryptophan, tyrosine, valine, ascorbic acid, biotine (H), carnitine, cholecalciferol, choline chloride, cyanocobalamine (B₁₂), ergocalciferol, folic acid, myo-inositol, menadione, nicotinamide, PABA, panthotenato, pyridoxal, pyridoxamine, pyridoxine, retinol (A), riboflavine (B₂), Thiamine (B₁), 6,8 Thiotic acid, alfa-tocoferol, 3-phytylmenadione (K₁), tetrahydrofolic acid, hemin from procine, and nanopure water.

### DETAILED DESCRIPTION OF THE INVENTION

The present invention concerns novel compositions for use as a growth medium for culturing parasitic organisms. The preferred embodiment described herein relates to the production of compositions comprising immunogenic polypeptides or the nucleic acids encoding them which can elicit an immune response in a warm-blooded animal, thereby inducing clinical remission of psoriasis, atoptic dermatitis, and other related immunological pathologies. One skilled in the art would recognize that the present invention can be used with any parasitic organism and the following description is meant to be illustrative and not limiting in the scope of the invention.

The subject polypeptides of the preferred embodiment are from Leishmania protozoa and, preferably, from killed Leishmania amastigote protozoa. The polypeptides of the subject invention can be obtained from protozoa of the Leishmania genus using standard protein isolation procedures which are known in the art. A first-generation polyvalent immunotherapeutic agent is provided, comprising a polypeptide isolate of a mixture of a plurality of Leishmania species, such as L. (L) amazonensis, L. (L) venezuelensis, L. (V) brasiliensis, L. (L) chagasi, L. (L) donovani, L. (L) infantum, L. (L) major, L. (L) panamensis, L. (L) tropica, and L. (L) guyanensis. Preferably, the mixture comprises L. (L) amazonensis, L. (L) venezuelensis, L. (V) brasiliensis, and L. (L) chagasi. Most preferably, the mixture consists of these four species.

In the prior art, the organisms were cultivated in the amastigote stage in the synthetic culture medium specified in Table 1, supplemented with 5% fetal bovine serum, typically at about 30-34° C. Subsequently, and during the stationary phase of growth, the amastigotes are subjected to a medium containing an amount of N-p-tosyl-L-Lysine chloromethyl ketone (TLCK) or a pharmacologically acceptable salt thereof effective to kill the cells. The dead cells are then isolated and treated with the non-ionic detergent Nonidet p-40 (NP40) to solubilize the surface antigens, which are discarded. The particulate antigens that comprise the immunogenic polypeptides of the present invention can be collected by centrifugation following cell disruption. These polypeptides are washed with phosphate-buffered saline (PBS) and subsequently resuspended by sonication for 5 minutes at 4° C. in PBS containing alumina.

One of ordinary skill in the art of molecular biology can obtain nucleic acids encoding the polypeptides. For example, the polypeptides of the first-generation immunotherapeutic agent have been isolated and purified from protozoa of the Leishmania genus and comprise eight bands, identified by SDS-PAGE, representing eight distinct polypeptides having apparent molecular weights of 21, 33, 44, 50, 55, 58, 65, and 77 kDa, respectively. Each of these bands represents a separate polypeptide that can be isolated and sequenced in accordance with standard amino acid sequencing procedures. The polypeptides of each second-generation immunotherapeutic agent were purified by subjecting the first-generation immunotherapeutic agent containing the mixture of eight polypeptides to chromatography on diethylaminoethyl(DEAE)-Sephadex. Two fractions having all the activity to cure psoriasis were isolated and totally reduced and alkylated by standard procedures. These fractions were subjected to electrophoresis on acrylamide gels to separate the constituent polypeptides, and the amino acid sequence of each polypeptide was obtained by standard protein sequencing procedures. The nucleotide sequences encoding each of these polypeptides can be derived from these amino acid sequences by application of the genetic code.

TABLE 1
Prior art Leishmania culture medium.

| Ingredient | mg/lt |
|---|---|
| Methionine | 140 |
| Tryptophan | 50 |
| α-Amino Adipic Acid | 3 |
| Asparagine | 165 |
| Cystine | 47 |
| Histidine | 6 |
| Aspartic Acid | 120 |
| Alanine | 512 |
| Proline | 248 |
| Lysine | 337 |
| Taurine | 6 |
| Isoleucine | 191 |
| Ornithine | 3 |
| Tyrosine | 210 |
| β-alanine | 80 |
| Phosphoserine | 23 |
| α-amino Butyric Acid | 8 |
| Leucine | 440 |
| Arginine | 413 |
| Serine | 220 |
| Hydroxylysine | 12 |
| Glutamine | 164 |
| Glutamic Acid | 420 |
| Cysteine | 0.5 |
| Phosphoethanolamine | 25 |
| Threonine | 200 |
| Glycine | 235 |
| Phenylalanine | 240 |
| Valine | 266 |
| d-Pantothenic Acid | 1 |
| Ascorbic Acid | 0.05 |
| p-Aminobenzoic Acid | 0.05 |
| Ergocalciferol (D₂) | 0.1 |
| L-carnitine | 0.05 |
| DL-methionine-S-methyl- | 0.05 |
| sulfonium chloride (U) | |
| 2-Deoxyadenylic acid | 3.0 |
| (d-AMP) | |
| 5′-Thymidylic Acid (TMP) | 3.0 |
| 2′Deoxycitidine-5- | 3.0 |
| monophosphate (d-CMP) | |
| Carnosine | 25 |
| Citrulline | 50 |
| Sarcosine | 57 |
| CaCl₂ | 265 |
| Fe(NO₃)9H₂O | 0.72 |
| KCl | 400 |
| MgSO₄ 7 H₂O | 200 |
| NaCl | 5,850 |
| NaHCO₃ | 2,000 |
| NaH₂PO₄H₂O | 140 |
| Tricine | 900 |
| Hemin | 1 |
| HEPES | 2,000 |
| Glucose | 1,000 |
| D-ribose | 10 |
| 2-Deoxy-ribose | 10 |
| Cholecalciferol(D₃) | 0.1 |
| Biotin | 1 |
| Pyridoxamine | 0.05 |
| Pyridoxal | 1 |
| Cyanocobalamin(B₁₂) | 0.01 |
| Choline | 1 |
| Thiamine (B₁) | 1 |
| Inositol | 2 |
| α-Tocopherol | 0.01 |
| 3-phytylmenadione(K₁) | 0.01 |
| Menadione (K₃) | 0.01 |
| Retinol (A) | 0.14 |
| Riboflavin (B₂) | 0.1 |
| 6,8 Thiotic Acid | 0.01 |
| Pyridoxine (B₆) | 0.025 |
| Folic Acid | 1 |
| Niacinamide | 1 |
| Tetrahydrofolic Acid | 0.5 |
| Adenosine-5-Triphosphate (ATP) | 5.5 |
| 2′-Deoxyuridine-5-monophosphate | 3.0 |
| (d-UMP) | |
| 5′-Deoxyguanylic Acid (d-GMP) | 3.0 |
| Hydroxyproline | 262.5 |

The present invention allows for the cultivation of the Leishmania in the amastigote stage in a culture media that is free of fetal bovine serum. The cultured amastigotes can then be used in the same fashion as outlined above in the production of immunotherapeutic or therapeutic agents. The preferred embodiment of the inventive culture media is described in Table 2. The constituents of the medium are mixed in a suitable vessel at room temperature. The porcine hemin is put in water, at 0.5 mg/ml concentration, then completely dissolved adding a few drops of concentrated NaOH and filtered by 0.2 mu filters.

TABLE 2
Leishmania culture medium

| Product | mg/lt |
|---|---|
| Calcium chloride | 265 |
| Sodium Bicarbonate | 2200 |
| Potasium chloride | 400 |
| Sodium Chloride | 6.8 |
| Monosodium Phosphate | 140 |
| Glucose | 2,000 |
| Hepes | 2,340 |
| Ferric Nitrate | 0.72 |
| Magnesium Sulfate | 200 |
| Tricine | 900 |
| D-Ribose | 10 |
| 2-Deoxy ribose | 10 |
| Adenosine-5-Triphosphate | 21.1 |
| (ATP) | |
| 2-Deoxyadenylic acid | 12 |
| (d-AMP) | |
| 5′-Thymidylic Acid (TMP) | 12 |
| 2′-Deoxicitidine-5 | 12 |
| monophosphate (d | |
| 2′-Deoxyuridine-5- | 12 |
| monophosphate (d | |
| 2′-Deoxyguanilic Acid | 12 |
| (d-GMP) | |
| Aspartic Acid | 120 |
| Glutamic Acid | 845 |
| L-Alanine | 512 |
| Arginine | 413 |
| Carnosine | 25 |
| Cysteine | 0.5 |
| Cystine | 47 |
| Glutamine | 164 |
| Glycine | 235 |
| Histidine | 6 |
| Iso-Leucine | 191 |
| Leucine | 440 |
| Lysine | 337 |
| Methionine | 140 |
| Ornitine | 36 |
| Phenylalanine | 240 |
| Proline | 248 |
| Serine | 220 |
| Threonine | 200 |
| Tryptophan | 50 |
| Tyrosine | 210 |
| Valine | 266 |
| Ascorbic Acid | 0.05 |
| Biotine (H) | 1 |
| Carnitine | 0.05 |
| Cholecalciferol | 0.1 |
| Choline chloride | 1 |
| Cyanocobalamine (B₁₂) | 0.01 |
| Ergocalciferol | 0.1 |
| Folic Acid | 1 |
| Myo-Inositol | 2 |
| Menadione | 0.01 |
| Nicotinamide | 1 |
| PABA | 0.05 |
| Panthotenato | 1 |
| Pyridoxal | 1 |
| Pyridoxamine | 0.05 |
| Pyridoxine | 0.025 |
| Retinol(A) | 0.14 |
| Riboflavine (B₂) | 0.1 |
| Thiamine (B₁) | 1 |
| 6,8 Thiotic acid | 0.01 |
| Alfa-Tocoferol | 0.01 |
| 3-phytylmenadione (K₁) | 0.01 |
| Tetrahydrofolic Acid | 0.5 |
| Hemin from Porcine | 1 |
| Nanopure Water | 1 |
| Qsd in litrs | |

The foregoing description of specific embodiments is merely illustrative, and various modifications may be made without deviating from the spirit and scope of the present invention, which is limited only by the following claims.

1. A growth medium for use with parasitic organisms, said media being free of bovine serum.

2. The medium of claim 1 wherein said media comprises calcium chloride, sodium bicarbonate, potassium chloride, sodium chloride, monosodium phosphate, glucose, hepes, ferric nitrate, magnesium sulfate, tricine, d-ribose, 2-deoxy ribose, adenosine-5-triphosphate (ATP), 2-deoxyadenylic acid (d-AMP), 5′-thymidylic acid (TMP), 2′-deoxyicitidine-5 monophosphate (d, 2′-deoxyuridine-5-monophosphate (d, 2′-deoxyguanilic Acid (d-GMP), aspartic acid, glutamic acid, 1-alanine, arginine, carnosine, cysteine, cystine, glutamine, glycine, histidine, iso-leucine, leucine, lysine, methionine, ornitine, phenylalanine, proline, serine, threonine, tryptophan, tyrosine, valine, ascorbic acid, biotine (H), carnitine, cholecalciferol, choline chloride, cyanocobalamine (B₁₂), ergocalciferol, folic acid, myo-inositol, menadione, nicotinamide, PABA, panthotenato, pyridoxal, pyridoxamine, pyridoxine, retinol (A), riboflavine (B₂), Thiamine (B₁), 6,8 Thiotic acid, alfa-tocoferol, 3-phytylmenadione (K₁), tetrahydrofolic acid, hemin from procine, and nanopure water.

3. A growth medium for use in the cultivation of Leishmania genus in the amastigote stage, said media being free of bovine serum.

4. The medium of claim 3 wherein said media comprises calcium chloride, sodium bicarbonate, potassium chloride, sodium chloride, monosodium phosphate, glucose, hepes, ferric nitrate, magnesium sulfate, tricine, d-ribose, 2-deoxy ribose, adenosine-5-triphosphate (ATP), 2-deoxyadenylic acid (d-AMP), 5′-thymidylic acid (TMP), 2′-deoxyicitidine-5 monophosphate (d, 2′-deoxyuridine-5-monophosphate (d, 2′-deoxyguanilic Acid (d-GMP), aspartic acid, glutamic acid, 1-alanine, arginine, carnosine, cysteine, cystine, glutamine, glycine, histidine, iso-leucine, leucine, lysine, methionine, ornitine, phenylalanine, proline, serine, threonine, tryptophan, tyrosine, valine, ascorbic acid, biotine (H), carnitine, cholecalciferol, choline chloride, cyanocobalamine (B₁₂), ergocalciferol, folic acid, myo-inositol, menadione, nicotinamide, PABA, panthotenato, pyridoxal, pyridoxamine, pyridoxine, retinol (A), riboflavine (B₂), Thiamine (B₁), 6,8 Thiotic acid, alfa-tocoferol, 3-phytylmenadione (K₁), tetrahydrofolic acid, hemin from procine, and nanopure water.

5. A method of maintaining a parasitic organism in vitro comprising contacting a culture comprising the parasitic organism with a bovine serum-free culture medium.

6. The method of claim wherein said culture free medium comprises: calcium chloride, sodium bicarbonate, potassium chloride, sodium chloride, monosodium phosphate, glucose, hepes, ferric nitrate, magnesium sulfate, tricine, d-ribose, 2-deoxy ribose, adenosine-5-triphosphate (ATP), 2-deoxyadenylic acid (d-AMP), 5′-thymidylic acid (TMP), 2′-deoxyicitidine-5 monophosphate (d, 2′-deoxyuridine-5-monophosphate (d, 2′-deoxyguanilic Acid (d-GMP), aspartic acid, glutamic acid, 1-alanine, arginine, carnosine, cysteine, cystine, glutamine, glycine, histidine, iso-leucine, leucine, lysine, methionine, ornitine, phenylalanine, proline, serine, threonine, tryptophan, tyrosine, valine, ascorbic acid, biotine (H), carnitine, cholecalciferol, choline chloride, cyanocobalamine (B₁₂), ergocalciferol, folic acid, myo-inositol, menadione, nicotinamide, PABA, panthotenato, pyridoxal, pyridoxamine, pyridoxine, retinol (A), riboflavine (B₂), Thiamine (B₁), 6,8 Thiotic acid, alfa-tocoferol, 3-phytylmenadione (K₁), tetrahydrofolic acid, hemin from procine, and nanopure water.

7. The method of claim 5 wherein said parasitic organism is a member of the Leishmania genus in the amastigote stage.

8. The method of claim 7 wherein said culture medium comprises: calcium chloride, sodium bicarbonate, potassium chloride, sodium chloride, monosodium phosphate, glucose, hepes, ferric nitrate, magnesium sulfate, tricine, d-ribose, 2-deoxy ribose, adenosine-5-triphosphate (ATP), 2-deoxyadenylic acid (d-AMP), 5′-thymidylic acid (TMP), 2′-deoxyicitidine-5 monophosphate (d, 2′-deoxyuridine-5-monophosphate (d, 2′-deoxyguanilic Acid (d-GMP), aspartic acid, glutamic acid, 1-alanine, arginine, carnosine, cysteine, cystine, glutamine, glycine, histidine, iso-leucine, leucine, lysine, methionine, ornitine, phenylalanine, proline, serine, threonine, tryptophan, tyrosine, valine, ascorbic acid, biotine (H), carnitine, cholecalciferol, choline chloride, cyanocobalamine (B₁₂), ergocalciferol, folic acid, myo-inositol, menadione, nicotinamide, PABA, panthotenato, pyridoxal, pyridoxamine, pyridoxine, retinol (A), riboflavine (B₂), Thiamine (B₁), 6,8 Thiotic acid, alfa-tocoferol, 3-phytylmenadione (K₁), tetrahydrofolic acid, hemin from procine, and nanopure water.
